(19) United States
(12) Patent Application Publication
(10) Pub. No.: US 2026/0355712 A1
(43) Pub. Date: Nov. 12, 2026

(54) FURNACE

(71) Applicant: INOVYN EUROPE LIMITED, Runcorn, Cheshire (GB)

(72) Inventors: Paul DEGRAEVE, Isieres (BE); Roel MILIS, Mechelen (BE)

(21) Appl. No.: 19/130,697

(22) PCT Filed: Nov. 16, 2023
(86) PCT No.: PCT/EP2023/082094

§ 371 Date: May 16, 2025

(30) Foreign Application Priority Data
Nov. 22, 2022 (EP) 22208938.5

(51) Int. Cl. B01J 19/24

(52) U.S. Cl. CPC B01J 19/2465; B01J 19/2425; B01J 2208/00265; B01J 2219/00135

(57) ABSTRACT

An electrically heated furnace, particularly a furnace for chemical reactions, the furnace includes a furnace cabin that includes one or more process tubes which extend through the furnace cabin, a plurality of electrically powered heating means located inside the furnace cabin for heating the one or more process tubes, one or more gas inlets, and one or more gas outlets. The furnace also includes an external recirculation loop which connects the one or more gas outlets of the furnace cabin to the one or more gas inlets of the furnace cabin such that gas exiting the one or more gas outlets of the furnace cabin flows to the one or more gas inlets and is recirculated to the furnace cabin, and at least one gas analyser or detector and/or at least one pressure monitor for measurement of pressure is provided on the external recirculation loop.

### FIELD AND BACKGROUND OF THE INVENTION

The present invention relates to an electrically heated furnace (and which as used herein may also be referred to a reactor), particularly a furnace for chemical reaction, for example for cracking of hydrocarbons such as ethane, naphtha and 1,2-dichloroethane. The present invention also relates to an electrically heated furnace used for heating-up petroleum or chemical products or for gas processing.

Furnaces for cracking of hydrocarbons such as ethane, naphtha, 1,2-dichloroethane and others are well-known. Historically, the reaction has been performed by passing the reactant hydrocarbon through an externally heated reaction tube located within a furnace to provide the heating for the endothermic cracking reaction. In traditional processes the heat required has been obtained by combustion of a fuel in burners located inside the furnace.

In traditional furnace, it is common to have one or more radiation sections in the lower part where burners deliver the heat to process tubes and a convection section where heat from hot combustion gases is transferred to process and/or utility tubes before their release to atmosphere. It has also been proposed to provide the heat for cracking by electrical heating, for example using heating elements located on the walls of a furnace or heating coils on the reaction tubes. Also direct electrical heating of the reaction tubes has been proposed.

Although the optimum design for an electrically heated cracking furnace would differ from that of conventional burner heated cracking furnaces, in some situations it would nevertheless be advantageous to retrofit an existing burner fired furnace to use electrical heating rather than build a new furnace from scratch. In particular, this would enable large parts of existing systems, including the process feed supply systems, the physical furnace structure, the process tubes and downstream quench systems to be used largely unchanged.

### SUMMARY OF THE INVENTION

We have now found an improved design for an electrically heated furnace, and in particular, whilst the design could also be applied to a “new build” is also suitable for retrofitting an existing cracking furnace.

Thus, in a first aspect, the present invention provides an electrically heated furnace, the furnace comprising:
a. A furnace cabin, the furnace cabin comprising:
   i. one or more process tubes which extend through the furnace cabin,
   ii. a plurality of electrically powered heating means located inside the furnace cabin for heating the one or more process tubes,
   iii. one or more gas inlets,
   iv. one or more gas outlets,
      characterised in that the furnace further comprises:
b. An external recirculation loop which connects the one or more gas outlets of the furnace cabin to the one or more gas inlets of the furnace cabin,
c. At least one gas analyser or detector and/or at least one pressure monitor for measurement of pressure provided on the external recirculation loop.

Further, in a second aspect, the present invention provides an electrically heated furnace, the furnace comprising:
a. A furnace cabin, the furnace cabin comprising:
   i. one or more process tubes which extend through the furnace cabin,
   ii. a plurality of electrically powered heating means located inside the furnace cabin for heating the one or more process tubes,
   iii. one or more gas inlets,
   iv. one or more gas outlets,
      characterised in that the furnace further comprises:
b. An external recirculation loop which connects the one or more gas outlets of the furnace cabin to the one or more gas inlets of the furnace cabin,
c. One or more heaters for heating the gas in the external recirculation loop.

The present invention, in both the first and second aspects, provides an electrically heated furnace.

The furnace comprises a furnace cabin, which comprises one or more process tubes which extend through the furnace cabin. In general, in use, reactants to be reacted are passed through the inside of the process tubes. The process tube or tubes are heated, and thereby the reactants passing through the process tube or tubes are heated and caused to react.

Furnaces of this general structure, albeit using other heating means, are known for a variety of different chemical processes, including for heating-up petroleum or chemical products or for gas processing. The process tube or tubes may, for example, each be a single straight tube which passes once through the furnace cabin. Alternatively, the process tube or tubes may, for example, have several straight sections within the furnace, linked by elbows or 180° bends e.g. to form a serpentine structure.

Particularly useful furnaces for the process of the present invention are furnaces for cracking of hydrocarbons such as ethane, naphtha and 1,2-dichloroethane.

As already noted, conventional furnaces for such reactions utilise burners located on the walls of the furnace cabin which burn a fuel to generate the heat required for heating the process tube or tubes. In the present invention the process tube or tubes are electrically heated. In particular, there are provided a plurality of electrically powered heating means located inside the furnace cabin for heating the one or more process tubes. Any suitable heating means may be provided. In one embodiment, the plurality of electrically powered heating means may be a plurality of electrically powered heating elements, in particular located on the walls inside the furnace cabin. In another embodiment, the plurality of electrically powered heating means may be a plurality of heating coils, in particular each being provided in the furnace cabin adjacent to one or more process tubes. In another embodiment, the plurality of electrically powered heating means may be means by which a process tube or tubes are directly electrically heated. Combinations of different heating means may also be used, including combinations of the above examples.

The furnace generally will comprise an electricity supply means located outside the furnace cabin for providing electricity to the electrically powered heating means. An issue with electrically heated furnaces is that this requires provision of electrical energy into a furnace which is not only at high temperatures, but where the external environment outside of the furnace is a hazardous “ATEX” area. “ATEX” generally refers to an area in which there is, or is a risk of, the presence of flammable gases which can give rise to the risk of explosion. An example of a suitable system can be found in WO 2021/214256, which provides movable current feed elements. More preferably, the electricity supply means may utilise one or more hermetically sealed boxes (and which as used herein may also be referred to as “ATEX boxes”) which boxes are connected externally to the furnace cabin. The electrical connection to the heating means may be provided by one or more electrical connecting rods or other connections which pass through the wall of the hermetically sealed box and of the furnace.

The furnace cabin in the present invention also comprises one or more gas inlets and one or more gas outlets. In general, there may be a plurality of inlets and/or a plurality of outlets.

The one or more gas inlets and one of more gas outlets provide, respectively, for introduction and removal of a gas from the furnace cabin, and in particular from to and from the volume of the furnace cabin which is outside of the one or more process tubes.

The one or more gas inlets and one or more gas outlets are preferably located on opposite sides of the furnace cabin. (“Sides” as used herein includes also the top and bottom.) This enables the gas to flow through the whole furnace cabin when passing from the one or more inlets to the one or more outlets. A plurality of inlets and/or outlets may be used, for example, to spread the gas flow across the volume of the furnace cabin. Alternatively, or in addition, however, baffles can also be used to optimise the gas flow through the furnace cabin.

In a most preferred embodiment the one or more gas inlets are located at the base of the furnace cabin and the one or more gas outlets are located at or close to the top of the cabin. In this manner the gas passes in an overall vertically upwards direction through the furnace cabin from the one or more gas inlets to the one or more gas outlets.

It will be noted that, especially in the preferred embodiment where the furnace is a retrofitted burner based furnace, that the furnace cabin may comprise a vent. As used herein, a vent is an outlet by which gas from the furnace cabin is vented to atmosphere or at least externally to the furnace. In conventional furnaces this may be a chimney, for example. In contrast, the one of more gas outlets of the present invention are outlets which are connected to an external recirculation loop. (The one or more gas outlets may be considered as recirculating gas outlets. Similarly, the one or more gas inlets may be considered as recirculating gas inlets.) The one of more gas outlets, in embodiments where there is also a chimney or other vent, are preferably provided on the main body of the furnace cabin and separate to any vent. However, whilst the one or more gas outlets are in addition to the vent, it is also within the scope of the present invention that one or more gas outlets may be taken from the base of the vent/chimney rather than directly from the furnace cabin or that a vent/chimney may be provided on the recirculation loop.

It is a key feature of the present invention that there is provided an external recirculation loop which connects one or more of the gas outlets of the furnace cabin to one or more of the gas inlets of the furnace cabin. In particular, the external recirculation loop provides a connection such that gas exiting the one or more gas outlets of the furnace cabin flows to the one or more gas inlets. It is thereby recirculated to the furnace cabin. More generally, in the furnace of the present invention gas flows through the furnace cabin, exits the furnace cabin through the one or more gas outlets, and then is returned through the external recirculation loop to the one or more gas inlets, and hence back into the furnace cabin. The gas which is recirculated may be herein referred to as the “recirculating gas”, and should be distinguished from the reactants and product gases (which may be referred to as “process gases”) in the one or more process tubes.

In the first aspect of the present invention there is provided at least one gas analyser or detector and/or at least one pressure monitor for measurement of pressure on the external recirculation loop. In use, therefore, gas is withdrawn from the furnace cabin through one or more gas outlets and enters the recirculation loop. Analysis and/or pressure measurement of the gas in the recirculation loop can be performed, whilst the gas circulates and passes through the one or more gas inlets back into the furnace cabin. It should be noted that those measurements are made on the gas in the recirculation loop, which is the gas from the furnace cabin. This is separate to any analysis on the reactants fed to or the products removed from the one or more process tubes.

In preferred embodiments there is provided at least one gas analyser or detector on the external recirculation loop. Optionally at least one pressure monitor may be provided in addition to the at least one gas analyser or detector in such embodiments.

In the second aspect of the present invention there are provided one or more heaters in the external recirculation loop for heating the gas in the external recirculation loop. In use, therefore, gas is withdrawn from the furnace cabin through one or more gas outlets and enters the recirculation loop. It is heated in the recirculation loop and then passes through the one or more gas inlets back into the furnace cabin.

In preferred embodiments there may be provided at least one gas analyser or detector and/or at least one pressure monitor for measurement of pressure on the external recirculation loop and also one or more heaters in the external recirculation loop for heating the gas in the external recirculation loop. Most preferably there is provided at least one gas analyser or detector on the external recirculation loop and one or more heaters in the external recirculation loop for heating the gas in the external recirculation loop.

In embodiments, there may be two or more recirculation loops. For example there may be a first recirculation loop by which one or more gas outlets are connected to one or more gas inlets, and a second recirculation loop by which one or more different gas outlets are connected to one or more gas inlets. In such embodiments at least one gas analyser or detector and/or at least one pressure monitor for measurement of pressure may be provided on one or both of the recirculation loops and/or one or more heaters may be present on one or both of the recirculation loops. It is preferred that one or more heaters are present on both recirculation loops. More preferably, however there is a single recirculation loop with all gas outlets combining to form a single pipe and which then feeds into the one or more gas inlets of the furnace cabin.

A number of advantages arise from the configurations of the present invention.

For example, the use of external heating allows additional heat input into the gas, and hence into the furnace cabin and to the process tubes. This can provide improved control of the energy input to the process. It also means that the gas entering the furnace cabin is hotter (than if external heating were not applied), so process tubes at or close to the one or gas inlets, for example at the base of the furnace cabin, receive more heat than they otherwise might. This can enable a more even heating of the furnace tubes overall, and better yield.

Further, in case of retrofit of existing burner fired furnaces, it is not always possible to supply the required heat duty of the furnace only by installing new electrical heating means within the existing cabin of the furnace. In such a case, the provision of additional heating in an external recirculation loop, allows to move part of the heat duty to the external recirculation loop.

In relation to the provision of an external recirculation loop with gas analysis/detection and/or pressure monitoring thereon, this is also advantageous. In particular, gas analysis is more easily done on the recirculation loop than trying to analyse gas inside the furnace cabin. The gas analysis on the recirculation loop can also more quickly identify any changes or issues in the gas medium in the furnace cabin. In particular, a relatively high recirculation loop flow (relative to the volume of the furnace cabin) can be used to ensure that the furnace cabin atmosphere is effectively sampled. For example, the volumetric flow rate in the recirculation loop (“recirculation rate”) may be from 2 to 100% of the furnace cabin volume per minute (i.e. for a volume of X m³, the flow rate may be between 0.02 times X m³/min and X m³/min). This means that the gas in the furnace cabin has an average residence time of 1 to 50 minutes. Where one or more heaters for heating the gas are provided in the external recirculation loop then the optimum recirculation flow rate will generally be selected based on the desired temperature for the recirculating gas after heating. In addition, or alternatively, a plurality of gas outlets can be used such that the recirculating gas is representative of the atmosphere in multiple parts, if not all, of the furnace cabin. In contrast, if analysers are provided in the furnace cabin itself then multiple analysers may be required for effective coverage of the furnace volume. (A typical furnace cabin volume for a conventional furnace for cracking of 1,2-dichloroethane may, for example, be from 200 to 500 m³. Steam cracking furnaces for cracking of ethane may be of a similar size or even bigger. Effectively sampling all areas of such furnaces requires multiple analysers.)

In embodiments, the gas in the furnace cabin may be circulating within the furnace cabin itself (as well as in the external recirculation loop). This is advantageous as it also means that an individual gas outlet can sample gas circulating from other parts of the furnace cabin rather than from a single static location, and this can be achieved even without a relatively large volumetric flow rate in the external recirculation loop. This can also allow to more quickly identify any changes or issues in the gas medium throughout the furnace cabin, therefore. (In such embodiments the volumetric flow rate in the recirculation loop (“recirculation rate”) may be in the ranges previously noted, although in such cases it is preferably at the lower end of the ranges previously noted, such as from 2 to 25% of the furnace cabin volume per minute.

However, particularly if efficient circulation is obtained within the furnace itself, the volumetric flow rate in the recirculation loop (“recirculation rate”) may be lower than 2% of the furnace cabin volume per minute. Thus, in embodiments in which the gas in the furnace cabin is circulating within the furnace cabin itself, the volumetric flow rate in the recirculation loop (“recirculation rate”) may be in the range 0.5 to 25%, such as 0.5 to 5%, of the furnace cabin volume per minute.) Circulation within the furnace cabin in such embodiments may be achieved by any suitable means. This can include one or more of: one or more fans inside the furnace cabin, one or more baffles inside the furnace cabin and/or by provision and location of multiple gas inlets for the recirculating gas entering the furnace cabin from the external recirculation loop to provide flow currents in the furnace cabin, for example. In most preferred embodiments, circulation within the furnace cabin is achieved, at least in part, by convection currents within the furnace cabin. For example, providing heating on the walls of a furnace cabin can lead to gas which rises at the walls of the furnace cabin (as it is heated), and then cools and sinks in the centre of the furnace cabin, where is contacts (and heats) process tubes, leading to circulation by convection currents.

More specifically, it is known in conventional cracking furnaces that process tubes can rupture. This can cause hydrocarbons or other reactants and products to enter the furnace cabin. In conventional furnaces these mix with a hot burner combustion product stream which generally comprises CO2 and O2 (because excess O2 is added to ensure complete combustion) and is then vented. In the present invention the gas is recirculated and the concentration of leaking components can increase rapidly. Analysis of the recirculating gas in the recirculation loop can therefore quickly identify even a small leakage of hydrocarbons or other reactants/products into the furnace cabin. The most relevant analyser or detector will depend on the process. It could be, for example, an analyser or detector for detection of hydrocarbons, ammonia, hydrogen or hydrogen chloride. In a process for the production of ethylene dichloride (EDC) an EDC or an HCl analyser or detector is preferably provided. (As used herein, an analyser will provide a concentration, whereas a detector provides an alert if the component is detected.)

In general the recirculating gas may be an inert gas (like N2, CO2, argon) or may include oxygen, such as being air or a gas mixture of an inert gas with 1-21% oxygen. A small amount of oxygen in the furnace cabin, such as 1-10 vol % oxygen, may, for example, be desirable for the optimum operation of the electric heating means. Thus, in preferred embodiments an O2 analyser or detector is also provided. The presence of O2 (e.g. when using an inert gas) or a deviation from the expected oxygen content (e.g. when using a gas comprising oxygen) could signal a leak of external air into the system. This could be, for example, a leak into the furnace cabin or a leak into the recirculation loop.

In some embodiments there may be provided a pressure monitor on the external recirculation loop. Usually the reactants in the process tubes are at a much higher pressure than the gas in the furnace cabin. Thus, a deviation in pressure on the external recirculation loop (and in particular a pressure increase) may also be used as an indication of a leak from the process tubes. If the furnace cabin is itself at a pressure higher than atmospheric, which is preferred in some embodiments, then a reduction in pressure may indicate a leak from the furnace cabin to the external environment.

The recirculating gas may include steam.

The analysis/detection/monitoring thus provides significant safety advantages by detecting potentially dangerous situations rapidly. A reading which indicates a deviation from the expected value, for example of hydrocarbon, HCl or oxygen, would then trigger a safety response. The response would be selected by the person skilled in the art but would generally depend on the specific gas detected and the extent thereof. For example, a small oxygen increase may need to be monitored but operation could potentially continue. In contrast, a leak of a reactant or product, such as (depending on the process) a hydrocarbon leak, an HCl leak or an ammonia leak would result in stopping of the process. Various actions may be taken in this scenario, including purging of the furnace cabin and/or flushing of the process tubes, as would be apparent to a person skilled in the art.

In general (i.e. the first or second aspects), to provide the necessary recirculation the furnace may comprise, in the recirculation loop, a suitable fan or blower, or gas ejector. Fresh gas for the furnace cabin/recirculation loop may be introduced in either the furnace cabin (by one or more separate fresh gas inlets) or in the recirculation loop. Where an ejector is used then the fresh gas is suitably introduced into the ejector as the motive gas.

In the second aspect of the present invention there are provided one or more heaters for heating the gas in the external recirculation loop. In the first aspect it is also preferred that there are provided one or more heaters for heating the gas in the external recirculation loop.

The following description therefore applies to the second aspect and also to those embodiments of the first aspect where one or more heaters are provided for heating the gas in the external recirculation loop.

The one or more heaters for heating the gas in the external recirculation loop may be any suitable heaters. However, the present invention is particularly advantageous where the external heating is also provided by one or more electrical heating means. As with the heating in the furnace cabin, any suitable electrical heating means may be provided.

Typically, in use, most of the overall heat (i.e. more than 50%) which is required to be supplied is provided from the electrical heating elements in the furnace cabin. Thus, usually, the installed heating capacity of the electrical heating elements in the furnace cabin (“internal” heating or heating capacity) is larger than the installed heating capacity in the heaters for heating the gas in the external recirculation loop (“external” heating or heating capacity). (As used herein “installed heating capacity” means the amount of heating power which is available. This is independent of whether, in use, all this capacity is used for heating.)

Typically, in a retrofitted furnace, the size of the furnace cabin can limit the number of heating elements which can be provided in the furnace cabin, and hence the heating capacity which can be installed. In such embodiments, the installed heating capacity in the furnace cabin is maximised (taking into account the constraint of it size), and sufficient external heating provided to provide the desired total heating capacity for the furnace.

In typical embodiments, the installed heating capacity in the furnace cabin is 50-95% of the total installed heating capacity (i.e. total heat capacity of the electrically powered heating means located inside the furnace cabin and the one or more heaters for heating the gas in the external recirculation loop), such as 60-90% of the total installed heating capacity.

In absolute terms, the total available installed heat capacity may be selected as required, not least based on the feed to be heated and its throughput i.e. the scale of the process. It may be, for example, 5 to 200 MW, such as 5 to 100 MW. The installed heat capacity of the electrically powered heating means located inside the furnace cabin may be 4 to 175 MW and the typical installed heat capacity of the one or more heaters for heating the gas in the external recirculation loop may be 1 to 100 MW.

In one embodiment the total available installed heat capacity may be 5 to 60 MW, with the installed heat capacity of the electrically powered heating means located inside the furnace cabin being 4 to 50 MW and the installed heat capacity of the one or more heaters for heating the gas in the external recirculation loop being 1 to 30 MW. In most preferred embodiments, the total available installed heat capacity may be 5 to 30 MW, with the installed heat capacity of the electrically powered heating means located inside the furnace cabin being 4 to 25 MW and the installed heat capacity of the one or more heaters for heating the gas in the external recirculation loop being 1 to 15 MW.

However, despite the above typical ranges, there is no fundamental reason why the installed heating capacity in any external heaters cannot be larger than the installed heating capacity in the furnace cabin and/or in use, why all of the heat applied cannot come from external heaters.

More generally, in use, the ratio of heat delivered by the external heaters in the external recirculation loop to the total heat delivered by the furnace (internal+external heating) may be from 5 to 100%. The preferred ratio, however, is 5 to 50% i.e. preferably most of the total heat is delivered by the electrical heating elements in the furnace cabin, not by the external heaters.

In general, the present invention provides the ability to adjust this ratio as required. The total amount of heating applied, and the relative heating applied in the furnace cabin and in the recirculation loop may be adjusted, for example, based on the feedstock to be cracked.

In one embodiment, the recirculation loop includes a box or a pipe or of several pipes which are conveying the recirculating gas and which contain electrically powered heating elements or coils located on the internal walls or distributed within the gas flow. Heating elements or coils may be equipped with fins to improve heat transfer.

In another embodiment, the recirculation loop is made of one tube or of multiple tubes in parallel who are externally heated in a box containing electrically powered heating element. This external box may be of much simpler design than the furnace cabin since no reactants or products are present.

In another embodiment, the recirculation loop is made of one tube or of multiple tubes in parallel who are directly electrically heated.

### BRIEF DESCRIPTION OF DRAWINGS

FIG. 1 is a schematic depiction of an electrically heated furnace.

FIG. 2 is a schematic depiction of a furnace formed by fitting electrical heating elements and a recirculation loop to a conventional fired furnace cabin.

### DETAILED DESCRIPTION OF THE INVENTION

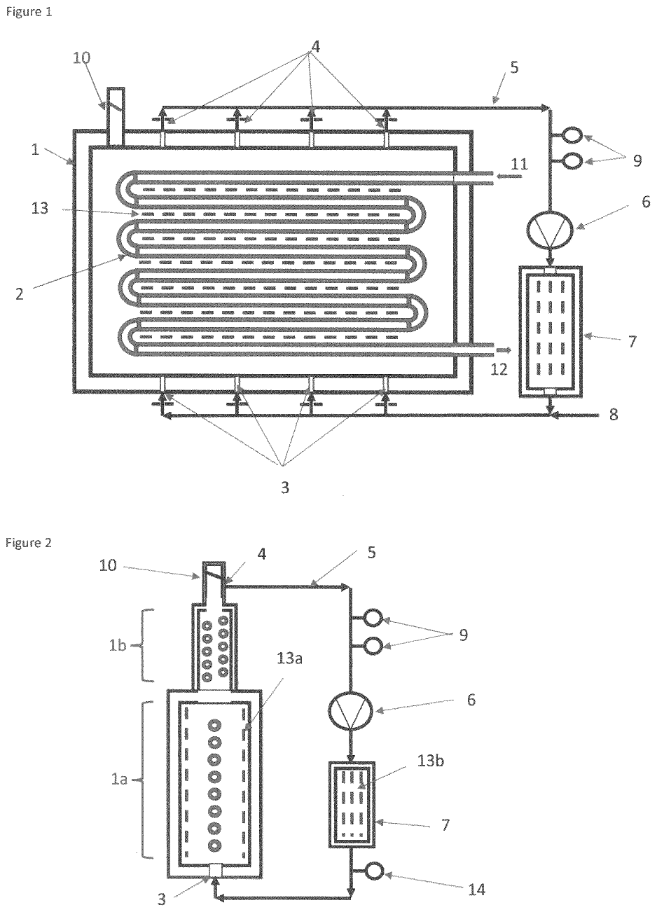

One example of a particularly preferred configuration is shown in FIG. 1. In particular, FIG. 1 shows schematically an electrically heated furnace comprising a furnace cabin (1) with a serpentine process tube (2) and heating elements (13). The furnace cabin has gas inlets (3) and gas outlets (4)—four of each are shown but this may be more or less. Gas removed from the one or more gas outlets is passed through a recirculation loop (5) using a circulation fan (6). The gas is heated in an external heating box (7), which is also electrically heated. Fresh gas is introduced via line 8, whilst there are provided analysers (9), for example for analysis of HCl and O2. The furnace further comprises a chimney (10), whilst reactant gas is introduced to the process tube (2) via an inlet (11), with products being removed via an outlet (12).

Another example of a particularly preferred configuration is shown in FIG. 2. In particular, FIG. 2 shows schematically a furnace formed by fitting electrical heating elements and a recirculation loop (5) to a conventional fired furnace cabin. This type of configuration could be used for retrofitting/conversion of an existing gas fired furnace, enabling the utilisation of the existing furnace cabin and process tubes, as well as feed controls and product work-up. As shown, the furnace cabin comprises a conventional gas fired furnace cabin with a radiation section (1a) and a convection section (1b). The gas fired heaters (burners) in the radiation section have been replaced with electrical heating elements (13a). Process tubes pass reactant gas to and through the convection section (1b) and then the radiation section (1a), as in the conventional/existing furnace arrangement. The furnace cabin has a gas inlet (3) and a gas outlet (4). Gas removed from the gas outlet (4) is passed through a recirculation loop (5) using a circulation fan (6). The gas is heated in an external heating box (7) for heating gas in the recirculation loop before it is recycled to the furnace cabin via the inlet (3). The heating may in particular use further electrical heating elements (13b) in the external heating box (7). Analysers (9), for example for analysis of HCl and O2, are provided on the recirculation loop (5), along also with a pressure monitor (14). Although not shown, an inlet can be provided on the recirculation loop for introduction of fresh inert gas or air.

Whilst FIGS. 1 and 2 both illustrate a furnace which includes both analysers and external heating on the external recirculation loop, it will be appreciated that the external recirculation loop may comprise solely one or more analysers or solely heaters for heating the gas in the external recirculation loop, and still be according to the respective first and second aspects of the present invention.

The furnace of the first aspect or the second aspect of the present invention may be used for any process which typically operates in furnaces in which reactants or process fluids are passed through heated process tubes, including any processes conventionally or historically operated in fired (burner based) furnaces.

Thus, in a third aspect, the present invention provides a process for performing a chemical reaction which process comprises
a. providing a furnace according to the first aspect and/or according to the second aspect of the invention,
b. passing one or more reactants through the one or more process tubes,
c. circulating a gas through the one or more gas outlets, the recirculation loop, the one or more gas inlets and the furnace cabin, and
d. heating the reactants in the one or more process tubes to effect reaction of the reactants within the process tubes.

The temperature of the reaction will depend on the specific process and is not especially limited, but in preferred processes the furnace may typically operate at a temperature of the process tubes in the range 300 to 1200 °C. The process may be catalytic or non-catalytic. In the former case, catalysts may be provided in the process tubes either as a catalyst bed or as a coating on the inside of the process tubes.

Examples of suitable processes include steam and other cracking processes, various reforming processes, such as steam reforming and dry reforming, processes for the dehydrogenation of alkanes.

In particularly preferred embodiments, the process is a process for cracking, and most particularly a process for cracking of 1,2-dichloroethane (EDC) to produce vinyl chloride monomer (VCM). The cracking of EDC to produce VCM is well-known in the art. The present invention, similar to operation in a conventional process operates in a furnace by passing an EDC containing stream through a process tube inside the furnace cabin, and heating the tube to heat and crack the EDC therein.

Typically, and preferably, the (or each if more than one) process tube in such a furnace is in the form of a serpentine tube which is located in a vertical plane or close to a vertical plane in the centre of the furnace cabin. Heating can then be applied from heating elements on both sides of the furnace cabin.

The EDC containing stream is heated to a temperature sufficient to cause cracking of the EDC. Typically this is at least 350 °C, and preferably in the range 350 °C to 550 °C. The temperatures, residence times etc. may be selected by the person skilled in the art for the degree or rate of cracking required. They may, in particular, be similar to cracking in conventional (hydrocarbon burner) systems.

The EDC containing stream may be introduced in a form where the EDC is in the liquid phase. In this case the EDC is vaporised in the earlier sections of the process tube or tubes, then further heated to a temperature, for example within the range 350 °C to 550 °C, at which cracking occurs in later sections. In other embodiments the EDC containing stream introduced at the inlet may have been heated (“pre-heated”) externally to the furnace, for example to a temperature sufficient to vaporise any liquid EDC. In such a case the EDC containing stream is in the gaseous phase at the inlet of the process tube(s).

The furnace may however also be designed for and used for other processes, such as steam cracking. The furnace may also be designed for heating-up petroleum or chemical products.

The furnace may comprise one or more heaters for heating the gas in the external recirculation loop. As already noted, the ratio of heat delivered by any external heaters in the external recirculation loop to the total heat delivered by the furnace (internal+external) may be from 5 to 100%. The preferred ratio, however, is 5 to 50% i.e. preferably most of the total heat is delivered by the electrical heating elements in the furnace cabin, not by the external heating elements.

As also already noted, the present invention provides the ability to adjust this ratio as required. The total amount of heating applied, and the relative heating applied in the furnace cabin and in the recirculation loop may be adjusted, for example, based on the feedstock to be cracked. (And this may be, for example, based on different hydrocarbon feeds, for example ethane versus naphtha cracking may have different requirements, or the “state” of the feed on entry to the process e.g. whether EDC is liquid or gaseous on entry to the furnace.)

As noted, the (recirculating) gas may be an inert gas or a gas comprising oxygen. In general an inert atmosphere is preferred, although a small amount of oxygen (for example, a gas mixture which comprises an inert gas and less than 10 vol % oxygen) may be desirable for the use of certain types of electrical heating elements.

The furnace cabin maybe operated above atmospheric pressure. This is advantageous in relation to potential external contamination with air because it ensures that the gas in the furnace cabin leaks “out” rather than air leaking “in”.

### EXAMPLE

The present invention can be illustrated by the following Example, which illustrates the retrofitting of a conventional (burner based) EDC (ethylene dichloride) cracker. The retrofitted furnace is as shown in FIG. 2.

The EDC cracker produces 160 kt/y of VCM (vinyl chloride monomer). EDC feed is 53 t/h of EDC, which is fed to the convection section of the cracker, and then passes to the radiation section.

Cracking rate is close to 60%.

Prior to retrofitting the thermal cracker is heated up by natural gas burners distributed on several rows on the lateral walls of the radiation section of the cracking furnace. Flat type flame burners are heating up the process tubes in the radiation section. Flue gases are then passing through the convection section of the furnace. At the top of the cracker, flue gases are removed through the chimney and either sent to atmosphere or to an independent heat recovery step.

The required heat duty of the burners is 17.9 MW. A significant part of this heat leaves the cracker in the flue gases.

The electrical cracker is as shown in FIG. 2, and is equipped with an external recirculation loop. This loop recirculates gases from top of the cracker (top of convection section) back into the radiation section, via an external electrical heating section. Analysers are provided on the recirculation loop for analysis of HCl and O₂. These constantly analyse the recirculating gas stream. In particular, the presence of HCl or a change in O₂ concentration would rapidly identify the presence of a leak from a process tube.

In this example, nickel-chromium-aluminium heating elements are used as electrical heating means in both the radiation section of the cracking furnace and in the external heating section. To crack 53 t/h of EDC there is provided 9.8 MW of heat from the electrical heating elements inside the radiation section of the cracking furnace, and 4.1 MW of heat from the external electrical heating section. The circulating gas exits the convection section at 450 °C (which is the same as the exiting flue gas in the “conventional” furnace) and is heated to 800 °C in the external heating section before it is reintroduced to the radiation section. The recirculation gas flow is close to 30000 Nm³/h (500 Nm³/min).

Compared with the burner based operation the total heat input (4.1+9.8 MW) for the electrical cracker configuration is significantly reduced because the large flow of hot flue gas exiting the process is eliminated. (In particular, in the process according to the present invention only approximately 0.5 MW of energy is lost to heat losses.)

1. An electrically heated furnace, the furnace comprising:
a. A furnace cabin, the furnace cabin comprising:
i. one or more process tubes which extend through the furnace cabin,
ii. a plurality of electrically powered heating means located inside the furnace cabin for heating the one or more process tubes,
iii. one or more gas inlets, and
iv. one or more gas outlets,
b. An external recirculation loop which connects the one or more gas outlets of the furnace cabin to the one or more gas inlets of the furnace cabin such that gas exiting the one or more gas outlets of the furnace cabin flows to the one or more gas inlets and is recirculated to the furnace cabin, and
c. At least one gas analyser or detector and/or at least one pressure monitor for measurement of pressure provided on the external recirculation loop.

2. The electrically heated furnace according to claim 1, wherein the furnace further comprises one or more heaters for heating the gas in the external recirculation loop.

3. An electrically heated furnace, the furnace comprising:
a. A furnace cabin, the furnace cabin comprising:
i. one or more process tubes which extend through the furnace cabin,
ii. a plurality of electrically powered heating means located inside the furnace cabin for heating the one or more process tubes,
iii. one or more gas inlets, and
iv. one or more gas outlets,
b. An external recirculation loop which connects the one or more gas outlets of the furnace cabin to the one or more gas inlets of the furnace cabin such that gas exiting the one or more gas outlets of the furnace cabin flows to the one or more gas inlets and is recirculated to the furnace cabin, and
c. One or more heaters for heating the gas in the external recirculation loop.

4. The electrically heated furnace according to claim 2, wherein the one or more heaters for heating the gas in the external recirculation loop are one or more further electrical heating means.

5. The electrically heated furnace according to claim 1, wherein the plurality of electrically powered heating means located inside the furnace cabin comprises a plurality of electrically powered heating elements located on the walls inside the furnace cabin.

6. The electrically heated furnace according to claim 1, wherein the plurality of electrically powered heating means located inside the furnace cabin are means by which the one or more process tubes are directly electrically heated.

7. The electrically heated furnace according to claim 1, wherein furnace cabin has a plurality of gas inlets and/or a plurality of gas outlets.

8. The electrically heated furnace according to claim 1, wherein the one or more gas inlets are located at a base of the furnace cabin and the one or more gas outlets are located at or close to a top of the cabin.

9. The electrically heated furnace according to claim 1, wherein the furnace cabin comprises, in addition to the one or more gas outlets, a vent by which gas from the furnace cabin is vented to atmosphere or externally to the furnace.

10. The electrically heated furnace according to claim 1, wherein at least one gas analyser or detector for detection of one of hydrocarbons, ammonia, hydrogen, oxygen or hydrogen chloride is provided on the external recirculation loop.

11. The electrically heated furnace according to claim 10, wherein an analyser or detector for detection of hydrogen chloride or detection of ethylene dichloride (EDC) is provided on the external recirculation loop.

12. The electrically heated furnace according to claim 10, wherein two or more analysers or detectors are provided on the external recirculation loop, at least one of the analysers or detectors being an analyser or detector for the detection of oxygen.

13. The process according to claim 16, wherein a volumetric flow rate in the external recirculation loop is from 2 to 100% of a furnace volume per minute.

14. The process according to claim 16, wherein the gas in the furnace cabin is circulating within the furnace cabin itself, this circulation being caused by at least one of the group consisting of: one or more fans inside the furnace cabin, one or more baffles inside the furnace cabin, by provision and location of multiple gas inlets for the recirculating gas entering the furnace cabin from the external recirculation loop to provide flow currents in the furnace cabin, and by convection currents within the furnace cabin.

15. The process according to claim 14, wherein a volumetric flow rate in the external recirculation loop is from 0.5 to 25% of a furnace volume per minute.

16. A process for performing a chemical reaction which process comprises:
a. providing a furnace according to claim 1,
b. passing one or more reactants through the one or more process tubes,
c. circulating a gas through the one or more gas outlets, the external recirculation loop, the one or more gas inlets and the furnace cabin, and
d. heating the one or more reactants in the one or more process tubes to effect reaction of the one or more reactants within the one or more process tubes.

17. The process according to claim 16, wherein the furnace operates at a temperature of the one or more process tubes in a range of 300 to 1200 °C.

18. The process according to claim 16, wherein the process is a process for cracking of 1,2-dichloroethane (EDC) to produce vinyl chloride monomer (VCM), and wherein an EDC containing stream is heated in the one or more process tubes to a temperature in a range of 350 °C. to 550 °C.

19. The process according to claim 16, wherein there is circulated in the furnace cabin and the recirculation loop an inert gas or a gas mixture which comprises an inert gas and less than 10 vol % oxygen.
